(19) United States
(12) Patent Application Publication
(10) Pub. No.: US 2026/0130867 A1
(43) Pub. Date: May 14, 2026

(54) FORMULATION

(71) Applicant: ORPHALAN S.A., Paris (FR)

(72) Inventor: Naseem Amin, London (GB)

(21) Appl. No.: 19/382,917

(22) Filed: Nov. 7, 2025

(30) Foreign Application Priority Data
Nov. 8, 2024 (EP) 24306883.0
Jul. 21, 2025 (EP) 25306182.4

(51) Int. Cl. A61K 31/132; A61K 9/20; A61K 9/28

(52) U.S. Cl. CPC A61K 31/132; A61K 9/2009; A61K 9/2013; A61K 9/2018; A61K 9/284

(57) ABSTRACT

This invention relates to pharmaceutical formulations comprising triethylenetetramine tetrahydrochloride (TETA·4HCl). Specifically, it relates to pharmaceutical compositions which are suitable for once a day administration to a subject in need of treatment.

### CROSS REFERENCE TO RELATED APPLICATIONS

The present application claims priority to EP Application No. 25306182.4, filed Jul. 21, 2025, and EP Application No. 24306883.0, filed Nov. 8, 2024, the entire disclosure of each of which is hereby incorporated by reference.

### FIELD OF THE INVENTION

This invention relates to pharmaceutical formulations comprising triethylenetetramine tetrahydrochloride (TETA·4HCl). Specifically, it relates to pharmaceutical compositions which are suitable for once a day administration to a subject in need of treatment.

### BACKGROUND OF THE INVENTION

Triethylenetetramine, or 1,2-ethanediamine, N, N′-bis(2-aminoethyl) (also known as trientine, or TETA) has the structure:

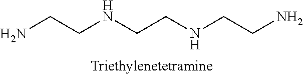

Trientine is a polyamine chelator of copper (II). Its copper chelating properties make it useful in the treatment of various conditions, in particular Wilson's disease (also known as Wilson disease). Wilson's disease is a genetic disorder caused by a mutation in the Wilson disease protein (ATP7B gene). The condition leads to a build up of copper in the body. The copper chelating ability of trientine has also led to its consideration for the treatment of numerous conditions such as internal organ damage in diabetes patients, Alzheimer's disease and cancer (Henriet et al, International Journal of Pharmaceutics 511 (2016) 312-321). The tetrahydrochloride salt of trientine (TETA·4HCl) is a particularly advantageous salt of trientine for the treatment of Wilson's disease, but the dihydrochloride salt (TETA·2HCl) is also used.

Trientine (whether as the tetrahydrochloride or dihydrochloride) is typically administered to a subject in need of treatment several times a day, in order to better control copper levels. However, administration of several doses per day can be challenging over a lifetime, often leading to poor patient compliance due to strict timing requirements around the administration of treatment in relation to food, particularly for asymptomatic or stable conditions such as maintenance therapy for Wilson's disease (WD) (Ala et al, Dig. Dis. Sci 60 (2015) 1433-1439). Once daily administration of trientine would be advantageous to improve patient compliance by minimising the considerable challenge WD patients currently experience in adhering to treatment requirements around the intake of food or other co-medications during the day. Improved adherence to WD therapy may decrease disease progression and improve treatment outcomes for patients with Wilson's disease.

However, despite this desire for developing a trientine formulation which is suitable for once daily administration, so far no once daily administration formulation has been developed and used in a clinical setting for the treatment of Wilson's disease. This is at least partly due to challenges in developing suitable oral dosage forms. In particular, most existing oral dosage forms for trientine contain only up to around 200 mg trientine base, and so patients would be required to take a large number of dosage forms in one go in order to meet the maximum recommended dosage of up to around 1,500 mg/day. Formulations of trientine with higher amounts of trientine are known, but these are very large oral dosage forms (specifically capsules) which are difficult for patients to swallow. For example, TETA 2HCl capsules containing 250 mg or 500 mg TETA 2HCl (corresponding to around 167 mg or 333 mg trientine base, respectively) are known (National Drug Codes 16571-810-01, 16571-812-05). The 250 mg capsule is a “size 1” capsule, which is 19.4 mm in length and 6.9 mm in diameter. In contrast, the 500 mg capsule which contains twice the amount of active ingredient is a “size 00” capsule, which is 23.3 mm in length and 8.6 mm in diameter.

Furthermore, tablet formulations are generally preferred over capsule formulations, because they generally show greater storage stability, and can more easily be divided into multiple doses. However, increasing the amount of active ingredient in a tablet without significantly increasing the size of the tablet is not straightforward. Reformulation of the tablet is required, in order to create a formulation which contains, overall, less excipient to allow for more active ingredient, but whilst still including enough excipients, and in appropriate amounts, to provide acceptable pharmaceutical properties: such attributes include tabletting properties during manufacture, such as breakability (or hardness), friability and strength, as well as pharmacokinetic properties such as bioavailability (e.g. as measured using area under the curve) and dissolution rate/disintegration time. In particular, the physicochemical interactions of the active ingredient with the excipients can make it challenging to obtain such acceptable pharmaceutical properties at high loadings of the active ingredient. For example, not all copper antagonists that might be suitable for the treatment of Wilson's Disease can be formulated at high loadings of active ingredient whilst still retaining the acceptable pharmaceutical properties described above.

In order to improve patient compliance with once daily dosing there is therefore a need to provide oral dosage forms of trientine with higher amounts of active ingredient, in order to reduce the number of dosage forms that need to be taken at the same time, but without a significant increase in size of the dosage form (palatability) and which retains optimal properties for pharmaceutical administration.

### SUMMARY OF THE INVENTION

The present inventors have surprisingly found that triethylenetetramine tetrahydrochloride can be formulated into a tablet dosage form with very high levels of triethylenetetramine tetrahydrochloride (i.e. up to 95 wt %, relative to the total tablet weight), whilst still retaining optimal (or even improved) properties for pharmaceutical administration (for example size, breakability, bioavailability/area under the curve and dissolution rate/disintegration time), via the use of specific formulation ingredients. This formulation is therefore more useful for once a day administration than known formulations of trientine dihydrochloride or tetrahydrochloride, which contain at most around 50 wt % of trientine hydrochloride salt. Furthermore, this formulation is advantageously not significantly larger in size than known tablet formulations of TETA 4HCl, and is significantly smaller than known TETA 2HCl capsules which deliver similar amounts of active ingredient.

In particular, the present inventors have found that tablet dosage forms containing up to 95 wt % triethylenetetramine tetrahydrochloride can be made by reducing the amount of filler in the composition while still providing rapid dissolution and bioavailability. More specifically, the inventors have found that direct compression tablets with up to 95 wt % TETA 4HCl can be made whilst still retaining manageable size and performance in bioavailability, disintegration, and mechanical strength. This supports once-daily administration with improved compliance for Wilson's disease patients and enables dose individualization (e.g., pediatric dosing through tablet breaking). Clinical pharmacokinetic studies show AUC and Cmax values for once-daily administration are substantially better than for twice-daily forms, with no observed safety concerns.

The present invention therefore provides a tablet dosage form comprising triethylenetetramine tetrahydrochloride in an amount of from 60 to 95 wt %; and a first lubricant in an amount of from 0.1 to 10 wt %; wherein wt % amounts are relative to the total tablet weight.

The present invention also provides a tablet dosage form as described herein for use in preventing or treating Wilson's disease in a subject.

### DESCRIPTION OF THE FIGURES

FIG. 1: Image of a tablet core containing 85 wt % TETA 4HCl (relative to weight of tablet core).

FIG. 2: Image of a coated tablet containing 82.2 wt % TETA 4HCl (relative to total tablet weight).

FIG. 3: Study design for bioavailability study, comparing the new once a day formulation with the original twice a day formulation.

### DETAILED DESCRIPTION OF THE INVENTION

### Definitions

The formulations of the invention comprise triethylenetetramine tetrahydrochloride, also referred to herein as trientine tetrahydrochloride or TETA·4HCl.

Tablet dosage forms are referred to herein as tablets.

As used herein, the term “about” refers to the stated value±5%, preferably the stated value±2%.

Unless otherwise stated, all percentages mentioned herein are weight percentages (wt %). Wt % amounts are stated relative to the total tablet weight. In the case of a coated tablet, the total tablet weight is the weight of the coated tablet.

As used herein, and unless otherwise specified, reference to a weight of “triethylenetetramine” or “triethylenetetramine active ingredient” in a composition refers to the weight of triethylenetetramine free base in that composition. Where the weight of a salt of triethylenetetramine is intended (in particular a weight of triethylenetetramine tetrahydrochloride), this is explicitly specified.

### TETA·4HCl Tablets

The present invention provides tablet dosage forms comprising triethylenetetramine tetrahydrochloride in an amount of from 60 to 95 wt % relative to the total tablet weight. Preferably, the triethylenetetramine tetrahydrochloride is present in the tablet in an amount of from 70 to 95 wt % (for example, from 75 to 95 wt %, or from 76 to 95 wt %, or from 77 to 95 wt %, or from 78 to 95 wt %, or from 79 to 95 wt %, or from 80 to 95 wt %), more preferably from 70 to 90 wt %, more preferably from 75 to 90 wt % (for example, from 76 to 90 wt %, or from 77 to 90 wt %, or from 78 to 90 wt %, or from 79 to 90 wt %), yet more preferably from 80 to 90 wt %, and further preferably from 80 to 85 wt %.

The triethylenetetramine tetrahydrochloride may be present in the tablet in an amount of about 600 mg, thus providing a dosage of triethylenetetramine active ingredient (i.e. the triethylenetetramine base) of 300 mg (the remaining weight being hydrochloride salt). Alternatively, the triethylenetetramine tetrahydrochloride may be present in the tablet in an amount of about 100 mg to about 600 mg (i.e. about 50 mg to about 300 mg of triethylenetetramine base), for example about 100 mg to about 400 mg (i.e. about 50 mg to about 200 mg of triethylenetetramine base) or about 100 mg to about 200 mg (i.e. about 50 mg to about 100 mg of triethylenetetramine base). Tablets containing lower amounts of triethylenetetramine tetrahydrochloride may be particularly suitable for paediatric use.

The triethylenetetramine tetrahydrochloride may be provided in any suitable form. For example, the triethylenetetramine tetrahydrochloride may be crystalline triethylenetetramine tetrahydrochloride. Triethylenetetramine tetrahydrochloride is known to exist in at least two different polymorphic forms, known as Form A and Form B, as described in, for example, WO 2019/211464. Form A and Form B can be characterised by their XRPD pattern (for example measured using Cu-Kα X-ray radiation, which has a characteristic wavelength (k) of 1.5418 Å), as described in WO 2019/211464. Form A and Form B can alternatively, or additionally, be characterised by their Raman spectra. Form A and Form B may alternatively be characterised by any of the additional techniques described in WO 2019/211464, for example FTIR analysis.

For example, Form A triethylenetetramine tetrahydrochloride is typically characterised by an XRPD pattern having peaks at 25.2 and 35.7±0.1° 2θ, typically at 25.2 and 35.7±0.05° 2θ. Preferably the XRPD spectrum of Form A also has peaks at 21.8, 26.9 and 28.2±0.1° 2θ, typically at 21.8, 26.9 and 28.2±0.05° 2θ.

Form B triethylenetetramine tetrahydrochloride is typically characterised by an XRPD pattern having at least two peaks, at least three peaks, at least four peaks, at least five peaks, or all of the peaks, selected from the peaks at 22.9, 25.4, 25.8, 26.6, 34.6 and 35.3±0.1° 2θ. More preferably, Form B has an XRPD pattern having at least two peaks, preferably at least three, four, five or all of the peaks, selected from the peaks at 22.9, 25.4, 25.8, 26.6, 34.6 and 35.3±0.05° 2θ. It is particularly preferred that Form B has an XRPD pattern having peaks at 25.4, 34.6 and 35.3±0.1° 2θ, more preferably at 25.4, 34.6 and 35.3±0.05° 2θ.

The tablet dosage form may comprise triethylenetetramine tetrahydrochloride Form A. For example, the tablet dosage form may comprise from 60 to 95 wt % or from 70 to 95 wt % triethylenetetramine tetrahydrochloride Form A, preferably from 70 to 90 wt %, more preferably from 75 to 90 wt %, yet more preferably from 80 to 90 wt %, further preferably from 80 to 85 wt % triethylenetetramine tetrahydrochloride Form A. In one embodiment, the tablet dosage form comprises triethylenetetramine tetrahydrochloride Form A, and does not contain triethylenetetramine tetrahydrochloride Form B.

The tablet dosage form may comprise crystalline triethylenetetramine tetrahydrochloride Form B. For example, the tablet dosage form may comprise from 60 to 95 wt % or from 70 to 95 wt % triethylenetetramine tetrahydrochloride Form B, preferably from 70 to 90 wt %, more preferably from 75 to 90 wt %, yet more preferably from 80 to 90 wt %, further preferably from 80 to 85 wt % triethylenetetramine tetrahydrochloride Form B. In one embodiment, the tablet dosage form comprises triethylenetetramine tetrahydrochloride Form B, and does not contain triethylenetetramine tetrahydrochloride Form A.

The tablet dosage form may comprise a mixture of triethylenetetramine tetrahydrochloride Form A and Form B. For example, the tablet dosage form may comprise from 60 to 95 wt % or from 70 to 95 wt % of a mixture of triethylenetetramine tetrahydrochloride Form A and Form B, preferably from 70 to 90 wt %, more preferably from 75 to 90 wt %, yet more preferably from 80 to 90 wt %, further preferably from 80 to 85 wt % of a mixture of triethylenetetramine tetrahydrochloride Form A and Form B.

Where the tablet dosage form comprises a mixture of triethylenetetramine tetrahydrochloride Form A and Form B, the mixture may contain Form A and Form B in any relative amount. The mixture may contain no more than 50 wt % Form A, wherein wt % in this context refers to the weight expressed as the total amount of triethylenetetramine tetrahydrochloride in the tablet dosage form. Typically, the mixture contains no more than 40 wt % Form A, preferably no more than 30 wt % Form A, more preferably no more than 20 wt % Form A and most preferably no more than 10 wt % Form A. In other words, the mixture typically contains at least 50 wt % Form B (wherein wt % in this context refers to the weight expressed as the total amount of triethylenetetramine tetrahydrochloride in the tablet dosage form), preferably at least 60 wt % Form B, more preferably at least 70 wt % Form B, yet more preferably at least 80 wt % Form B, and most preferably at least 90 wt % Form B. However, in one embodiment, the mixture may contain at least 60 wt % Form A, for example at least 70 wt % Form A, at least 80 wt % Form A, or at least 90 wt % Form A. The mixture may contain no more than 40 wt % Form B, for example no more than 30 wt % Form B, no more than 20 wt % Form B, or no more than 10 wt % Form B.

The tablet dosage form comprises a first lubricant. The first lubricant may be any lubricant suitable for use in a tablet dosage form, and such lubricants are known to the person skilled in the art. The first lubricant may be selected from a fatty acid or a derivative thereof, a surfactant, an inorganic material or a polymer. The first lubricant may be selected from a metal stearate or a salt thereof (for example, magnesium stearate, calcium stearate, zinc stearate, or sodium stearyl fumarate), stearic acid, talc, magnesium aluminium silicate, sodium benzoate, a poly(ethylene oxide) and poly(propylene oxide) co-polymer (for example, Poloxamer 188), polyethylene glycol, sodium dodecyl sulfate, and vegetable oil. Preferably, the first lubricant is a metal stearate. More preferably, the metal stearate is one or more selected from magnesium stearate, calcium stearate and zinc stearate. Most preferably, the metal stearate is magnesium stearate. Preferably, the first lubricant is not glyceryl dibehenate. Preferably, the first lubricant is not stearic acid. In some embodiments, the tablet dosage form does not comprise stearic acid.

The tablet dosage form comprises the first lubricant in an amount of from 0.1 to 10 wt %. The tablet dosage form may comprise the first lubricant in an amount of from 0.1 to 8 wt %, for example from 0.1 to 5 wt %, or from 0.1 to 4 wt %, or from 0.1 to 3 wt %, or from 0.1 to 2 wt %, or from 0.1 to 1 wt %. Preferably, the tablet dosage form comprises the first lubricant in an amount of less than 1 wt %, and more preferably less than 0.5 wt %. Preferably, the tablet dosage form comprises the first lubricant in an amount of from 0.1 to 0.5 wt % (for example from 0.1 to 0.4 wt %), more preferably from 0.1 to 0.3 wt %. Most preferably, the tablet dosage form comprises the first lubricant in an amount of from 0.15 to 0.25 wt %.

In some embodiments, the first lubricant is selected from talc and magnesium aluminium silicate, and the first lubricant is present in an amount of from 0.1 to 10 wt %, for example from 0.1 to 8 wt %, or from 0.1 to 5 wt %, or from 0.1 to 3 wt %, or from 0.1 to 2 wt %. In some embodiments, the first lubricant is selected from stearic acid, a metal stearate, sodium benzoate, a poly(ethylene oxide) and poly(propylene oxide) co-polymer, polyethylene glycol, sodium dodecyl sulfate and vegetable oil, and the first lubricant is present in an amount of from 0.1 to 5 wt %, for example from 0.1 to 3 wt %, or from 0.1 to 2 wt %, or from 0.1 to 1 wt %. Preferably, the first lubricant is a metal stearate which is present in an amount of from 0.1 to 0.3 wt %. More preferably, the first lubricant is magnesium stearate which is present in an amount of from 0.1 to 0.3 wt %. The tablet may comprise from 0.7 to 2.0 mg magnesium stearate, for example from 1.0 to 1.5 mg magnesium stearate.

The tablet dosage form typically further comprises one or more fillers. For example, the tablet dosage form may comprise one, two or three fillers. Preferably, the tablet dosage form comprises one or two fillers, and more preferably one filler. The one or more fillers may be present in the tablet in an amount of from 5 to 39.9 wt % or from 5 to 29.9 wt % relative to the total tablet weight. Preferably, the one or more fillers are present in the tablet in an amount of from 5 to 25 wt %, more preferably from 5 to 20 wt %, yet more preferably from 5 to 15 wt %, and further preferably from 9 to 15 wt % relative to the total tablet weight. Previously known tablet dosage forms typically contain higher amounts of filler than 39.9 wt %, because higher amounts of filler have previously been thought to be necessary for providing the required binding of ingredients to prevent disintegration during tablet formation. However, it has been found that TETA 4HCl can be made into a pharmaceutically acceptable tablet with much lower amounts of filler.

The one or more fillers may be any suitable filler known in the art for making pharmaceutical formulations. Typically, the one or more fillers comprise mannitol, lactose, cellulose, microcrystalline cellulose, calcium carbonate, sorbitol, and/or starch. Preferably, the one or more fillers comprise mannitol, lactose, cellulose, microcrystalline cellulose, sorbitol, and/or starch. More preferably, the one or more fillers comprise mannitol. In one embodiment, the one or more fillers is mannitol. In some embodiments, the tablet dosage form comprises mannitol.

The tablet dosage form may comprise the one or more fillers in an amount of from 5 to 39.9 wt % or from 5 to 29.9 wt %, preferably from 5 to 25 wt %, more preferably from 5 to 20 wt %, yet more preferably from 5 to 15 wt %, and further preferably from 9 to 15 wt % relative to the total tablet weight, wherein the one or more fillers comprise mannitol. Preferably, the tablet dosage form comprises mannitol in an amount of from 5 to 39.9 wt % or from 5 to 29.9 wt %, preferably from 5 to 25 wt %, more preferably from 5 to 20 wt %, yet more preferably from 5 to 15 wt %, and further preferably from 9 to 15 wt % relative to the total tablet weight.

Preferred tablet dosage forms according to the invention include:

A tablet dosage form comprising:
70 to 95 wt % triethylenetetramine tetrahydrochloride; and
a metal stearate in an amount of from 0.1 to 0.3 wt %.

A tablet dosage form comprising:
80 to 90 wt % triethylenetetramine tetrahydrochloride; and
a metal stearate in an amount of from 0.1 to 0.3 wt %.

A tablet dosage form comprising:
80 to 85 wt % triethylenetetramine tetrahydrochloride; and
a metal stearate in an amount of from 0.1 to 0.3 wt %.

A tablet dosage form comprising:
70 to 95 wt % triethylenetetramine tetrahydrochloride; and
magnesium stearate in an amount of from 0.1 to 0.3 wt %.

A tablet dosage form comprising:
80 to 90 wt % triethylenetetramine tetrahydrochloride; and
magnesium stearate in an amount of from 0.1 to 0.3 wt %.

A tablet dosage form comprising:
80 to 85 wt % triethylenetetramine tetrahydrochloride; and
magnesium stearate in an amount of from 0.1 to 0.3 wt %.

A tablet dosage form consisting of:
70 to 95 wt % triethylenetetramine tetrahydrochloride;
0.1 to 0.3 wt % magnesium stearate;
5 to 29.9 wt % mannitol; and
0 to 10 wt % one or more further excipients,
wherein wt % amounts are relative to the total tablet weight.

A tablet dosage form consisting of:
80 to 90 wt % triethylenetetramine tetrahydrochloride;
0.2 to 0.3 wt % magnesium stearate;
5 to 15 wt % mannitol; and
0 to 10 wt % one or more further excipients,
wherein wt % amounts are relative to the total tablet weight.

A tablet dosage form consisting of:
80 to 85 wt % triethylenetetramine tetrahydrochloride;
0.2 to 0.3 wt % magnesium stearate;
9 to 15 wt % mannitol; and
0 to 10 wt % one or more further excipients,
wherein wt % amounts are relative to the total tablet weight.

The tablet dosage form may comprise one or more further excipients in addition to the first lubricant and optionally one or more fillers. Typically, the one or more further excipients are present in an amount of from 0 to 10 wt % relative to the total tablet weight. Preferably, the one or more further excipients are present in an amount of from 2 to 9 wt %, more preferably from 3 to 7 wt % relative to the total tablet weight.

In one embodiment, the one or more further excipients are selected from one or more stabilizers; one or more second lubricants (in addition to the first lubricant); and/or one or more coating excipients (preferably, one or more film coating excipients). Preferably, the tablet dosage form comprises one or more stabilizers, and one or more second lubricants (in addition to the first lubricant). More preferably, the tablet dosage form comprises one or more stabilizers; one or more second lubricants (in addition to the first lubricant); and one or more film coating excipients.

The tablet dosage form may comprise the one or more stabilizers in an amount of from 0.1 to 0.5 wt % relative to the total tablet weight. Preferably, the tablet dosage form comprises the one or more stabilizers in an amount of from 0.2 to 0.4 wt % relative to the total tablet weight.

The one or more stabilizers may be any stabilizer suitable for use in a tablet dosage form, and such stabilizers are known to the person skilled in the art. Suitable stabilizers include, for example, silicon dioxide (preferably colloidal silicon dioxide), syrup, acacia, gelatin, tragacanth, polyvinylpyrrolidone (Povidone), methylcellulose, ethylcellulose, sodium carboxymethylcellulose, hydroxypropylmethylcellulose, and sucrose. Preferably, the one or more stabilizers is selected from silicon dioxide (preferably colloidal silicon dioxide), polyvinylpyrrolidone (Povidone), methylcellulose, ethylcellulose, sodium carboxymethylcellulose, hydroxypropylmethylcellulose, and sucrose. In some embodiments, the tablet dosage form does not comprise a cellulose derivative, such as methylcellulose, ethylcellulose, sodium carboxymethylcellulose, hydroxypropylmethylcellulose. In some embodiments, the tablet dosage form does not comprise microcrystalline cellulose. More preferably, the one or more stabilizers comprise silicon dioxide, most preferably colloidal silicon dioxide. Preferably, the one or more stabilizers is colloidal silicon dioxide. The tablet dosage form preferably comprises colloidal silicon dioxide in an amount of from 0.1 to 0.5 wt %, preferably from 0.2 to 0.4 wt %, relative to the total tablet weight.

The tablet dosage form may comprise the one or more second lubricants in an amount of from 0.5 to 2.5 wt % relative to the total tablet weight. Preferably, the tablet dosage form comprises the one or more second lubricants in an amount of from 1 to 2.5 wt %, more preferably from 1 to 2 wt %, relative to the total tablet weight.

The one or more second lubricants may be any lubricant suitable for use in a tablet dosage form, and such lubricants are known to the person skilled in the art. The one or more second lubricants may be selected from the lubricants described herein as the first lubricant. The first lubricant may be the same or different as the one or more second lubricants. Typically, the first lubricant is different to the one or more second lubricants. Typically, the one or more second lubricants comprise glyceryl dibehenate. Preferably, the one or more second lubricants is glyceryl dibehenate. The tablet dosage form preferably comprises glyceryl dibehenate. The tablet dosage form preferably comprises glyceryl dibehenate in an amount of from 0.5 to 2.5 wt %, preferably from 1 to 2.5 wt %, more preferably from 1 to 2 wt % relative to the total tablet weight. Preferably, the one or more second lubricants does not comprise a metal stearate, and more preferably does not comprise magnesium stearate.

The tablet dosage form may be a coated tablet. Sugar coatings or film coatings may be used, preferably film coatings. Coated tablets typically comprise one or more coating excipients, typically one or more film coating excipients. The tablet dosage form may comprise the one or more coating excipients (preferably one or more film coating excipients) in an amount of from 0.1 to 7.5 wt % relative to the total tablet weight. Preferably, the tablet dosage form comprises the one or more coating excipients (preferably one or more film coating excipients) in an amount of from 0.5 to 7 wt %, preferably from 1 to 6 wt %, more preferably from 2 to 5 wt % relative to the total tablet weight.

The one or more film coating excipients may be any film coating excipient suitable for forming a coated tablet, and such film coating excipients are known to the person skilled in the art. Typically, the one or more film coating excipients are selected from a cellulosic (such as hydroxypropyl methyl cellulose (hypromellose), hydroxypropyl cellulose, and/or ethyl cellulose), a vinyl (such as polyvinyl alcohol), an alginate, a polyhydric alcohol (such as polyethylene glycol, and/or glycerine), an acetate ester (such as triacetin, and/or triethyl citrate), a glyceride, an oil, a colorant or pigment (such as titanium dioxide, iron oxides, natural colorants), and a carbohydrate or sugar (such as lactose, lactose monohydrate, polydextrose, and/or starch). The one or more film coating excipients may comprise hypromellose, lactose monohydrate, titanium dioxide, iron oxide, polyethylene glycol and/or triacetin. Preferably, the one or more film coating excipients comprise hypromellose, lactose monohydrate, titanium dioxide, iron oxide, polyethylene glycol and triacetin.

The tablet dosage form may contain additional pharmaceutically acceptable excipients, other than those identified above, which are suitable for use in oral dosage forms. This includes, for example, carriers, for example gelatin, sucrose, kaolin, dicalcium phosphate, and sodium chloride; disaggregating agents, for example starch, alginic acid, alginates or sodium starch glycolate; solubilising agents, e.g. cyclodextrins or modified cyclodextrins; effervescing mixtures; dyestuffs; sweeteners; wetting agents, such as lecithin, polysorbates, laurylsulphates; flavouring agents such as peppermint, oil of wintergreen, cherry flavouring and the like; and, in general, non-toxic and pharmacologically inactive substances used in pharmaceutical formulations. Such pharmaceutical preparations may be manufactured in a known manner, for example, by means of mixing.

In some embodiments, the tablet dosage form does not comprise any pharmacologically active substance other than the triethylenetetramine tetrahydrochloride. In other words, in some embodiments the tablet dosage form comprises triethylenetetramine tetrahydrochloride as the sole pharmacologically active substance. For example, preferably the tablet dosage form does not comprise a hypoglycemic agent. Preferably, the tablet dosage form does not comprise an antihypertensive agent. More preferably, the tablet dosage form does not comprise a hypoglycemic agent and also does not comprise an antihypertensive agent. In some embodiments, the tablet dosage form contains only (i.e. consists of) the triethylenetetramine tetrahydrochloride plus one or more excipients, wherein the excipients include at least the first lubricant. In this context, the term “excipient” refers to any substance which is not a pharmacologically active substance (for example, the various fillers, lubricants, stabilizers and coatings described herein). In some embodiments, the tablet dosage form contains only (i.e. consists of) the triethylenetetramine tetrahydrochloride and first lubricant, as well as any of the above described optional one or more fillers, pharmaceutically acceptable excipients, and coatings.

In one embodiment, the tablet is a breakable tablet. A breakable tablet may be scored such that is can be easily manually broken along the score line. The breakable tablet typically has a hardness of less than 370 N or less than 360 N, preferably less than 350 N, more preferably less than 340 N, yet more preferably less than 330 N, further preferably less than 320 N, and most preferably less than 310 N. The tablet dosage form preferably has a hardness of at least 300 N, and more preferably greater than 300 N. In one embodiment, the tablet has a hardness of from 300 to 360 N, for example from 300 to 350 N, preferably from 300 to 340 N, more preferably from 300 to 330 N, yet more preferably from 300 to 320 N, and most preferably from 300 to 310 N. Hardness may be measured using the methods of Ph. Eur. 10.0, 2.9.8, or USP (2024)<1217>. For example, hardness may be determined by measuring the force needed to crush the tablet, where the hardness value is taken as a mean result over 10 tablets. Specific conditions suitable for measuring hardness are set out in further detail in Example 3.

The tablet preferably has a friability of less than 1%, for example less than 0.750%, less than 0.5%, less than 0.25%, or less than 0.2%. Preferably, the tablet has a friability of less than 0.15%. Friability may be measured using the methods of Ph. Eur. 10.0, 2.9.7 or USP (2024)<1216>. For example, friability may be determined by tumbling the uncoated tablets in a drum rotating at 25±1 rotation/minute. Specific conditions suitable for measuring friability are set out in further detail in Example 3.

The tablet preferably has a disintegration time of less than 15 minutes, for example less than 10 minutes, less than 7 minutes, or less than 5 minutes. Preferably, the tablet has a disintegration time of less than 3 minutes. Disintegration time may be measured using the methods of Ph. Eur. 10.0, 2.9.1 or USP (2024)<701>. For example, disintegration time may be recorded as the time taken for 6 tablets to disintegrate in a liquid medium at 35-39° C. Specific conditions suitable for measuring disintegration time are set out in further detail in Example 3.

The tablet dosage form may have a length L of from 13 to 19 mm, a width W of from 6 to 10 mm, and/or a height H of from 4 to 7 mm. Preferably, the tablet dosage form has a length L of from 14 to 18 mm, a width W of from 7 to 9 mm, and/or a height H of from 4.5 to 6.5 mm; more preferably a length L of from 15 to 17 mm, a width W of from 7.5 to 8.5 mm, and/or a height H of from 5 to 6 mm; most preferably a length L of from 15.5 to 16.5 mm, a width W of from 7.5 to 8.5 mm, and/or a height H of from 5 to 6 mm. Alternatively, the tablet dosage form may have a length L of from 4 to 15 mm, a width W of from 3 to 10 mm, and/or a height H of from 2 to 7 mm. Preferably, the tablet dosage form has a length L of from 5 to 12 mm, a width W of from 4 to 9 mm, and/or a height H of from 3 to 6 mm; more preferably a length L of from 5 to 10 mm, a width W of from 5 to 9 mm, and/or a height H of from 3 to 6 mm.

The tablet dosage form may be any suitable shape, for example round or elongate. Therefore, the tablet dosage form may have a maximum dimension of from 13 to 19 mm, preferably from 14 to 18 mm, more preferably from 15 to 17 mm, and most preferably from 15.5 to 16.5 mm. Alternatively, the tablet dosage form may have a maximum dimension of from 4 to 15 mm, preferably from 5 to 12 mm, more preferably from 5 to 10 mm. In the case of a round tablet, the maximum dimension is typically the diameter. In the case of an elongate tablet, the maximum dimension is typically the length.

### Methods of Manufacturing Tablet Dosage Forms

TETA·4HCl can be produced by techniques known in the art. For example, TETA free base is commercially available and can be converted to the crystalline TETA hydrate and isolated by routine methods. The TETA hydrate can be treated with aqueous HCl to provide the TETA·4HCl salt. Typically, the TETA·4HCl salt is isolated in crude form, which may be recrystallised, as desired.

TETA·4HCl can be obtained in crystalline form, including polymorphic forms known as Form A and Form B. Recrystallisation may be carried out by techniques previously described. For instance, TETA·4HCl in crystalline form can be obtained by an anti-solvent crystallisation process, typically by the addition of an anti-solvent to an aqueous solution of TETA·4HCl and collecting the resulting crystals. TETA·4HCl Form B may be obtained by recrystallisation at a reduced temperature of 15° C. or below. Suitable methods for obtaining TETA·4HCl are described in WO2019/211464.

The TETA·4HCl may be milled or granulated prior to formulating into the tablet dosage form.

A tablet dosage form may be made by combining the TETA·4HCl, optionally after milling or granulating, with the tablet core excipients (i.e. the excipients which are to be included in the uncoated tablet). Typically, the tablet core excipients comprise all excipients to be included in the tablet dosage form, other than coating excipients, i.e. the first lubricant, optional filler and optionally one or more further excipients, excluding than coating excipients. The TETA·4HCl and tablet core excipients may be formed into a tablet by compression or moulding. Compressed tablets may be prepared by compressing in a suitable machine the active agent in a free flowing form such as a powder or granules, mixed with the tablet core excipients. Moulded tablets may be made by moulding in a suitable machine a mixture of the powdered compound and tablet core excipients, moistened with an inert liquid diluent such as purified water.

In a preferred embodiment, the tablet is coated, for example by film coating or sugar-coating, preferably by film-coating.

In one preferred embodiment, the tablet is scored. Preferably, the scored tablet is a breakable tablet.

In one embodiment, the tablet is formulated so as to provide slow or controlled release of the active agent.

In one embodiment, the tablet dosage form is produced by (a) obtaining TETA·4HCl in pure crystalline form, for example using the methods described above, (b) optionally milling and/or granulating the crystals obtained, (c) combining the TETA·4HCl Form B with the tablet core excipients, and (d) optionally mixing the TETA·4HCl and the core excipients; (e) compressing the mixture to form a tablet; and (f) optionally sugar-coating or film-coating the tablet.

### Medical Uses

A therapeutically effective amount of a tablet dosage form of the invention is administered to a subject. It will be understood that the specific dose level for any particular subject will depend upon a variety of factors including the age, body weight, general health, sex, diet, time of administration, drug combination and the severity of the particular disease undergoing treatment. Optimum dose levels and frequency of dosing will usually be determined by clinical trial.

A typical daily dose is up to 25 mg of triethylenetetramine base per kg of body weight, for example from 0.001 to 25 mg of triethylenetetramine base per kg of body weight, preferably from 5 to 25 mg of triethylenetetramine base per kg of body weight according to the age, weight and conditions of the subject to be treated, the type and severity of the disease and the frequency and route of administration. Preferably, daily dosage levels are from 150 mg to 1500 mg of triethylenetetramine (i.e. triethylenetetramine base), preferably from 300 mg to 1200 mg of triethylenetetramine base, more preferably from 450 to 975 mg triethylenetetramine base. For paediatric purposes, daily dosage levels are typically from 150 mg to 600 mg of triethylenetetramine base. Triethylenetetramine is typically administered to the patient in a non-toxic amount. Based on the relative molecular weights of triethylenetetramine and triethylenetetramine tetrahydrochloride, the corresponding dosage levels of the triethylenetetramine tetrahydrochloride are approximately double those of the triethylenetetramine active ingredient.

The tablet dosage form of the invention may be administered to a subject once daily, or multiple times a day (for example, twice, three times, or four times daily). The tablet dosage form of the invention is particularly suitable for once daily administration to a subject. Therefore, in a preferred embodiment, the tablet dosage form is administered to the subject once a day. Where the tablet dosage form is administered to the subject once a day, multiple tablet dosage forms may be administered together (e.g. immediately after one another) in order to provide the desired total daily dose.

The invention also provides a tablet dosage form as defined herein for use in a method of treatment of the human or animal body by therapy. In particular the tablet dosage form of the invention is useful in reducing copper levels in a subject and/or reducing the toxic effects of copper retention in a subject. The tablet dosage form is therefore useful in the treatment of disorders and diseases associated with raised copper levels. In particular, the tablet dosage form is useful in the prevention or treatment of Wilson's disease. In one embodiment, the tablet dosage form is useful in the treatment of Wilson's disease. In a preferred embodiment, the tablet dosage form is useful in the treatment of Wilson's disease in a subject, wherein the tablet dosage form is administered to the subject once a day. In some embodiments, the tablet dosage form is useful in the treatment of Wilson's disease in a subject, wherein the subject has not previously undergone treatment for Wilson's disease (also known as a previously treatment naïve subject receiving first line therapy). In some preferred embodiments, the tablet dosage form is useful in the treatment of Wilson's disease in a subject, wherein the subject has not previously undergone treatment for Wilson's disease (i.e. is a previously treatment naïve subject), and wherein the tablet dosage form is administered to the subject once a day. In other embodiments, the tablet dosage form is useful in the treatment of Wilson's disease in a subject, wherein the subject has previously undergone treatment for Wilson's disease using an active agent other than triethylenetetramine tetrahydrochloride (also known as second-line therapy). In some preferred embodiments, the tablet dosage form is useful in the treatment of Wilson's disease in a subject, wherein the subject has previously undergone treatment for Wilson's disease using an active agent other than triethylenetetramine tetrahydrochloride, and wherein the tablet dosage form is administered to the subject once a day. Such active agents are well known to the person skilled in the art, and include triethylenetetramine dihydrochloride, D-penicillamine, and zinc salts (such as zinc acetate).

The subjects treated according to the present invention may be human or animal subjects, in particular humans or mammals, typically humans.

Also disclosed herein are methods of treating or preventing (preferably treating) Wilson's disease in a subject, the method comprising administering the tablet dosage form as described herein to said subject. Preferably, the method comprises administering the tablet dosage form once a day.

Also disclosed herein is the use of triethylenetetramine tetrahydrochloride in the manufacture of a medicament for treating or preventing (preferably treating) Wilson's disease in a subject, wherein the medicament is a tablet dosage form as described herein. Preferably, the tablet dosage form is administered to said subject once a day.

The tablet dosage form of the invention has optimal pharmacokinetic properties, and may provide particularly optimised and effective serum AUC, Cₘₐₓ and Tₘₐₓ when administered to a subject, particularly when administered once a day. In one embodiment, the AUC₀₋₂₄ (i.e. from 0 to 24 hours after administration) is from about 10,000 to about 18,000 ng·hr/mL, for example from about 12,000 to about 17,000 ng·hr/mL, or from about 13,000 to about 16,000 ng·hr/mL, or from about 14,000 to about 15,000 ng·hr/mL (when the daily dosage is 900 mg trientine base, administered once a day). In one embodiment, the AUC₀₋₄₈ (i.e. from 0 to 48 hours after administration) is from about 10,000 to about 19,000 ng·hr/mL, for example from about 12,000 to about 18,000 ng·hr/mL, or from about 14,000 to about 17,000 ng·hr/mL, or from about 15,000 to about 16,000 ng·hr/mL (when the daily dosage is 900 mg trientine base, administered once a day). In one embodiment, the AUC₀₋₄₈ of a tablet dosage form of the invention administered once to a subject is from 80-125% of the AUC₀₋₄₈ of the same dosage of a Cuprior tablet which is administered twice to a subject (in a single day), for example about 116%. In one embodiment, Cₘₐₓ of the tablet dosage form of the invention (when the daily dosage is 900 mg trientine, administered once a day) is from about 2,500 to about 4,500 ng/mL, for example from about 2,750 to about 4,250 ng/mL, or from about 3,000 to about 4,000 ng/mL, or from about 3,250 to about 4,000 ng/mL, or from about 3,500 ng/mL to about 4,000 ng/mL. In one embodiment, Tₘₐₓ of the tablet dosage form of the invention (when the daily dosage is 900 mg trientine base, administered once a day) is from about 0.5 hours to about 5 hours, for example from about 0.5 hours to about 4 hours, or from about 0.5 hours to about 3 hours, or from about 0.5 hours to about 2 hours, or from about 0.5 hours to about 1.5 hours. It will be appreciated that where a different dosage is administered to a subject, the AUC, Cₘₐₓ and Tₘₐₓ values will differ accordingly, but the values above are representative of the physicochemical properties when administered at a dosage of 900 mg trientine base, once a day. Alternative dosages which may be administered to a subject are described herein.

Particular aspects of the invention are set out below.
1. A tablet dosage form comprising:
   triethylenetetramine tetrahydrochloride in an amount of from 60 to 95 wt %; and
   a first lubricant in an amount of from 0.1 to 10 wt %;
   wherein wt % amounts are relative to the total tablet weight.
2. A tablet dosage form according to aspect 1, comprising:
   triethylenetetramine tetrahydrochloride in an amount of from 70 to 95 wt %; and
   a first lubricant in an amount of from 0.1 to 10 wt %;
   wherein wt % amounts are relative to the total tablet weight.
3. A tablet dosage form according to aspect 1 or 2, which comprises from 80 to 95 wt % triethylenetetramine tetrahydrochloride.
4. A tablet dosage form according to any one of the preceding aspects, which comprises from 80 to 90 wt % triethylenetetramine tetrahydrochloride.
5. A tablet dosage form according to any one of the preceding aspects, which comprises from 80 to 85 wt % triethylenetetramine tetrahydrochloride.
6. A tablet dosage form according to any one of the preceding aspects, wherein the triethylenetetramine tetrahydrochloride comprises no more than 50 wt % triethylenetetramine tetrahydrochloride Form A and at least 50 wt % triethylenetetramine tetrahydrochloride Form B, wherein the wt % refers to the weight expressed as the total amount of triethylenetetramine tetrahydrochloride in the tablet dosage form.
7. A tablet dosage form according to any one of the preceding aspects, which comprises from 70 to 95 wt %, preferably from 80 to 95 wt %, more preferably from 80 to 90 wt %, yet more preferably from 80 to 85 wt % triethylenetetramine tetrahydrochloride Form B.
8. A tablet dosage form according to any one of the preceding aspects, wherein the first lubricant is selected from a fatty acid or a derivative thereof, a surfactant, an inorganic material or a polymer.
9. A tablet dosage form according to any one of the preceding aspects, wherein the first lubricant is selected from talc and magnesium aluminium silicate.
10. A tablet dosage form according to aspect 9, wherein the tablet dosage form comprises the first lubricant in an amount of from 0.1 to 10 wt %.
11. A tablet dosage form according to any one of aspects 1 to 8, wherein the first lubricant is selected from stearic acid, a metal stearate, sodium benzoate, a poly(ethylene oxide) and poly(propylene oxide) co-polymer, polyethylene glycol, sodium dodecyl sulfate and vegetable oil.
12. A tablet dosage form according to aspect 11, wherein the tablet dosage form comprises the first lubricant in an amount of from 0.1 to 5 wt %.
13. A tablet dosage form according to any one of aspects 1 to 8, 11 or 12, wherein the first lubricant is a metal stearate.
14. A tablet dosage form according to aspect 13, wherein the tablet dosage form comprises the first lubricant in an amount of from 0.1 to 0.3 wt %.
15. A tablet dosage form according to aspect 13 or aspect 14, wherein the metal stearate is one or more selected from magnesium stearate, calcium stearate and zinc stearate.
16. A tablet dosage form according to any one of aspects 13 to 15, wherein the first lubricant is magnesium stearate.
17. A tablet dosage form according to aspect 16, which comprises from 0.7 to 2.0 mg magnesium stearate.
18. A tablet dosage form according to any one of the preceding aspects, further comprising one or more fillers in an amount of from 5 to 39.9 wt % relative to the total tablet weight and one or more further excipients in an amount of 0 to 10 wt % relative to the total tablet weight.
19. A tablet dosage form according to aspect 18, wherein the one or more fillers comprise mannitol, lactose, cellulose, microcrystalline cellulose, calcium carbonate, sorbitol, and/or starch.
20. A tablet dosage form according to aspect 18 or aspect 19, wherein the one or more fillers comprise mannitol.
21. A tablet dosage form according to any one of aspects 18 to 20, which comprises from 5 to 15 wt % of one or more fillers.
22. A tablet dosage form according to aspect 21, which comprises from 9 to 15 wt % of one or more fillers.
23. A tablet dosage form according to aspect 1 consisting of:
   70 to 95 wt % triethylenetetramine tetrahydrochloride;
   0.1 to 0.3 wt % magnesium stearate;
   5 to 29.9% mannitol; and
   0 to 10 wt % one or more further excipients,
   wherein wt % amounts are relative to the total tablet weight.
24. A tablet dosage form according to aspect 1 consisting of:
   80 to 90 wt % triethylenetetramine tetrahydrochloride;
   0.2 to 0.3 wt % magnesium stearate;
   5 to 15 wt % mannitol; and
   0 to 10 wt % one or more further excipients,
   wherein wt % amounts are relative to the total tablet weight.
25. A tablet dosage form according to aspect 1 consisting of:
   80 to 85 wt % triethylenetetramine tetrahydrochloride;
   0.2 to 0.3 wt % magnesium stearate;
   9 to 15 wt % mannitol; and
   0 to 10 wt % one or more further excipients,
   wherein wt % amounts are relative to the total tablet weight.
26. A tablet dosage form according to any one of aspects 18 to 25, wherein the one or more further excipients are selected from one or more stabilizers; one or more second lubricants; and/or one or more film coating excipients.
27. A tablet dosage form according to aspect 26, wherein the tablet comprises the one or more second lubricants in an amount of from 0.5 to 2.5 wt % relative to the total tablet weight.
28. A tablet dosage form according to any one of aspects 26 or 27, wherein the one or more second lubricants is glyceryl dibehenate.
29. A tablet dosage form according to any one of aspects 26 to 28, wherein the tablet comprises the one or more stabilizers in an amount of from 0.1 to 0.5 wt % relative to the total tablet weight.
30. A tablet dosage form according to any one of aspects 26 to 29, wherein the one or more stabilizers is colloidal silicon dioxide.
31. A tablet dosage form according to any one of aspects 26 to 27, wherein the tablet is a coated tablet and comprises one or more film coating excipients in an amount of from 0.1 to 7.5 wt % relative to the total tablet weight.
32. A tablet dosage form according to any one of the preceding aspects which is a breakable tablet.
33. A tablet dosage form according to any one of the preceding aspects, wherein the tablet has a hardness of from 300 to 360 N.
34. A tablet dosage form according to aspect 33, wherein the breakable tablet has a hardness of from 300 to 310 N.
35. A tablet dosage form according to any one of the preceding aspects, wherein the tablet has a friability of less than 0.15%,
36. A tablet dosage form according to any one of the preceding aspects, wherein the tablet has a disintegration time of less than 5 minutes.
37. A tablet dosage form according to any one of the preceding aspects for use in preventing or treating Wilson's disease in a subject.
38. A tablet dosage form for use according to aspect 37, wherein the dosage form is administered to the subject once a day.

The following examples illustrate the invention but are not intended to limit the scope of the invention.

### EXAMPLES

### Reference Example 1: Known Tablet of TETA 4HCl

An example of a currently marketed coated tablet of TETA 4HCl is as described in Table 1.

TABLE 1
Currently marked coated tablet of TETA 4HCl

| | % w/w total |
|---|---|
| Component | tablet weight |
| TETA 4HCl | 48.1 |
| Mannitol | 46.3 |
| Magnesium stearate | 0 |
| Other excipients | 5.6 |

The average size of the coated tablet core is L 16.2 mm×W 8.2 mm×H 4.9 mm.

### Example 2: Manufacture of a Tablet with High Wt % of Active Ingredient

A 20 kg batch of film coated tablets was made according to the formulation in Table 2. All components were weighed according to the batch formulation and sieved through an oscillating sieve mill equipped with a stainless steel grid of 1 mm mesh size before loading.

16.99 kg of TETA 4HCl and 1.2 kg of mannitol were sieved then mixed for 10 min at 9 rpm into a 300 L stainless steel bin blender (tumble). 0.06 kg of silica colloidal anhydrous and 1.38 kg of mannitol were sieved then added to the premixture and additional mixing was performed for 10 min at 9 rpm. 0.32 kg of glycerol dibehenate and 0.04 kg Magnesium-Stearate were sieved then added to the mixture and the mixing restarted for 5 min at 9 rpm, until the final blend was a white powder without lumps. The mixture was directly compressed using a Fette 2090 rotary tablet press to yield capsule-shaped tablet cores (approximately 16×8 mm) with a break-mark on each side.

A suspension of Opadry® AMB II 88A120000 Yellow in purified water at 20% (w/w) was prepared in a stainless steel tank. The homogeneity of the film-coating suspension was checked visually before use. The film-coating suspension was sprayed onto the tablet cores in a stainless steel perforated pan coater (using an air inlet temperature of 65° C., at an air inlet volume of 3.8 m³/min, giving a calculated spray rate of 11 g/min). Upon completion of the spraying process, the film-coated tablets were discharged from the film coater.

TABLE 2
Formulation of tablets of the invention

| | Quantity | | % w/w |
|---|---|---|---|
| | per | % w/w | total |
| | tablet | tablet | tablet |
| Component | (mg) | core | weight |
| TETA 4HCl | 600.00 | 85.00 | 82.20 |
| Mannitol (Pearlitol 200 SD) | 91.07 | 12.90 | 12.48 |
| Colloidal (anhydrous) silica | 2.12 | 0.30 | 0.29 |
| dioxide (Aerosil 200) | | | |
| Glycerol dibehenate | 11.30 | 1.60 | 1.55 |
| (Compritol 888 ATO) | | | |
| Magnesium stearate | 1.41 | 0.20 | 0.19 |
| (Ligamed MF-2-V) | | | |
| Total | 705.90 | 100.00 | |
| Film coating excipients | 24.00 | | 3.29 |
| (Opadry AMB II yellow, | | | |
| 88A120000) | | | |
| Purified water | Removed during | | |
| | processing | | |
| Total | 729.90 | | 100.00 |

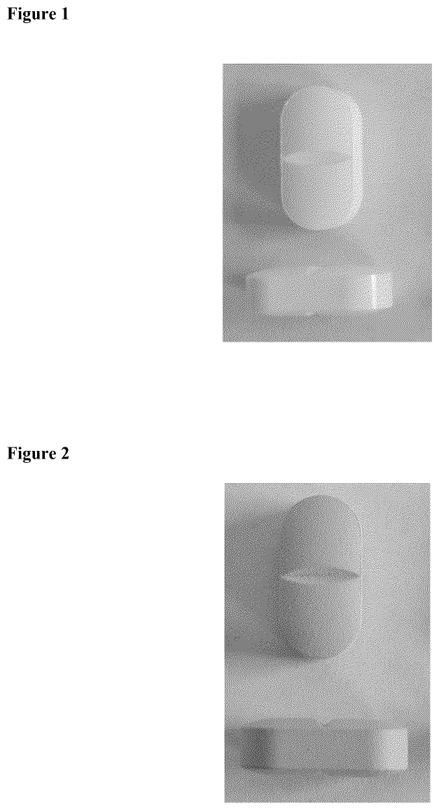

The average size of the coated tablet was L 16.2 mm×W 8.2 mm×H 5.4 mm. An image of a representative tablet core is provided in FIG. 1.

This example shows that it is possible to make a tablet dosage form containing much higher amounts of trientine active ingredient than previously used, for example up to 95 wt (relative to the total tablet weight).

### Example 3: Effect of the Amount of Magnesium Stearate in the Tablet

Additional tablet cores were made using the same process as Example 2, but with varying amounts of magnesium stearate, as shown in Table 3. Tablet cores A and B each had a height of 5.4 mm+0.1 mm. A film coating was then added according to the process in Example 2.

TABLE 3
Tablets with varying amounts of magnesium stearate

| | Tablet A | | | Tablet B | | |
|---|---|---|---|---|---|---|
| | | % w/w | | | % w/w | |
| | | total | % w/w | | total | % w/w |
| | Quan- | tablet | total | Quan- | tablet | total |
| | tity/ | weight | tablet | tity/ | weight | tablet |
| | tablet | (un- | weight | tablet | (un- | weight |
| | (mg) | coated) | (coated) | (mg) | coated) | (coated) |
| TETA 4HCl | 600.00 | 85.00 | 82.20 | 600.00 | 85.00 | 82.20 |
| Mannitol | 92.48 | 13.10 | 12.67 | 88.95 | 12.60 | 12.19 |
| (Pearlitol 200 | | | | | | |
| SD) | | | | | | |
| Colloidal | 2.12 | 0.30 | 0.29 | 2.12 | 0.30 | 0.29 |
| (anhydrous) | | | | | | |
| silica dioxide | | | | | | |
| (Aerosil 200) | | | | | | |
| Glycerol | 11.30 | 1.60 | 1.55 | 11.30 | 1.60 | 1.55 |
| dibehenate | | | | | | |
| (Compritol | | | | | | |
| 888 ATO) | | | | | | |
| Magnesium | 0.00 | 0.00 | 0.00 | 3.53 | 0.50 | 0.48 |
| stearate | | | | | | |
| (Ligamed MF- | | | | | | |
| 2-V) | | | | | | |
| Film coating | 24.00 | | 3.29 | 24.00 | | 3.29 |
| excipients | | | | | | |
| (Opadry AMB | | | | | | |
| II yellow, | | | | | | |
| 88A120000) | | | | | | |

The tablet cores (prior to film coating) were tested to assess their hardness, disintegration properties, and friability. The results are shown in Table 4.

Hardness was measured using the method of Ph. Eur. 10.0, 2.9.8. For each composition, 10 tablets were tested and the stated hardness value is a mean hardness from the 10 tablets.

Disintegration time was measured using the method of Ph. Eur. 10.0, 2.9.1. An apparatus was provided with a basket rack assembly with 6 tubes configured to move within a liquid material. One tablet was placed within each tube, and the liquid material was held at 35-39° C. The time taken for all 6 tablets to disintegrate was measured. A tablet was considered to be disintegrated when no residue of the tablet remains, or when any remaining residue was a soft mass having no palpably firm, unmoistened core.

Friability was measured using the method of Ph. Eur. 10.0, 2.9.7. Specifically, friability was measured using a tumbling drum with an internal diameter between 283-291 mm and a depth between 36-40 mm, of transparent synthetic polymer with polished internal surfaces, additionally containing a curved projection. A sample of whole uncoated tablets with a total mass of 6.5 g were dedusted, weighed and placed into the drum. The drum was rotated 100 times at 25±1 rotations/minute. The tablets were removed, loose dust was removed, and the tablets were weighed again.

TABLE 4
Properties of tablet cores with different
amounts of magnesium stearate

| | Tablet A | Example 2 | Tablet B |
|---|---|---|---|
| | (0 wt | (0.2 wt | (0.5 wt |
| | % MgSt) | % MgSt) | % MgSt) |
| Hardness (N) | 350-410 | 300-310 | 290-310 |
| Disintegration (min) | 2 | 2-3 | 3 |
| Friability (% 10 | 0.08 | 0.12 | 0.08 |
| tabl/.4 min) | | | |

### Conclusion

Tablet cores formed with over 80 wt % TETA 4HCl, but without any magnesium stearate, had a high hardness of 350 to 410 N (n=60). This high hardness meant that it was difficult to break the tablet along the score line by hand. In contrast, tablets including magnesium stearate had a lower hardness, which results in a more easily breakable tablet (which can be important in enabling dose adjustment).

Magnesium stearate is typically used in pharmaceutical formulations in an amount of from 0.25-5 wt % (Shangraw et al, Pharm. Technol., 17(1) (1993) 32-44). The use of 0.2 wt % magnesium stearate in Example 2 is associated with an unexpectedly significant impact on the hardness of the tablet compared with Tablet A (0 wt % MgSt), considering the presence of such a low amount of magnesium stearate. Further increasing the amount of magnesium stearate in Tablet B (0.5 wt % MgSt) does not provide further notable benefit.

Furthermore, the tablet core of Example 2 (0.2 wt % MgSt) showed a lower disintegration time than Tablet B (0.5 wt % MgSt), which is advantageous in pharmaceutical formulations to facilitate release of the active ingredient. This example therefore shows that the use of a low amount of 0.2 wt % MgSt is unexpectedly associated with optimal hardness and disintegration properties.

### Example 4: Comparison of Once a Day Administration Using Formulation of the Invention Vs Twice a Day Administration Using Known Formulation

A randomised, open-label, 2-way cross-over study was performed to evaluate and compare the PK, safety and tolerability of a total daily dose of 900 mg of TETA 4HCl administered once a day compared to twice a day.

### Method

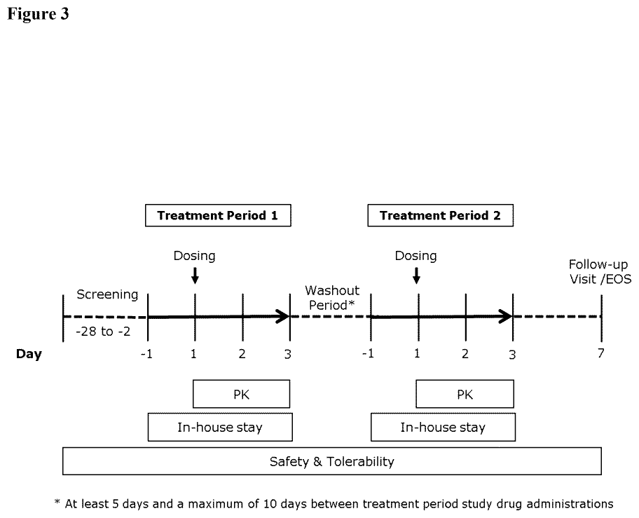

26 healthy subjects took part in the study. The cross-over study comprised a Treatment Period 1 and a Treatment Period 2 which each comprised a single dosage day, with the dosage days in each Treatment Period separated by a washout period of at least 5 days and a maximum of 10 days, as shown in FIG. 3.

In one of the Treatment Periods, the subject was administered 900 mg of trientine in a single administration (administered as 3×300 mg tablets according to Example 2). As outlined in Example 2, these tablets contain about 82 wt % TETA 4HCl (as a percentage of the coated tablet), and have a size of about L 16.2 mm×W 8.2 mm×T 5.4 mm. In the other Treatment Period, the subject was administered 450 mg of trientine twice in a single day, 8 hours apart (administered as 3×150 mg Cuprior® tablets, twice a day). Cuprior® tablets contain about 48 wt % TETA 4HCl (as a percentage of the coated tablet), and have a size of about L 16.2 mm×W 8.2 mm×T 4.9 mm.

For the once a day administration, serum samples were taken every hour for the first six hours following administration, then at hours 8, 12, 16, 20, 24, 36 and 48. For the twice a day administration, serum samples were taken every hour for the first six hours following the first administration, at hour 8 following the first administration (coinciding with administration of the second dose), every hour up until hour 14 (i.e. six hours after the second administration), and then at hours 16, 20, 24, 36 and 48. The serum samples were used to establish the concentration of trientine in the serum at each time point, as well as the concentration of two metabolites N(1)-acetyltriethylenetetramine (MAT) and N(1),N(10)-diacetyltriethylenetetramine (DAT).

### Results

No significant adverse effects were observed in either arm of the study. The results are shown in Tables 5 to 8.

TABLE 5
Results of bioavailability study

| | | | | | AUC (24 hr) | | AUC (48 hr) | |
|---|---|---|---|---|---|---|---|---|
| Dose | Tₘₐₓ (hr) | | Cₘₐₓ (ng/mL) | | (ng · hr/mL) | | (ng · hr/mL) | |
| (mg) | Mean | SD | Mean | SD | Mean | SD | Mean | SD |
| 900 mg | 1.1 | 0.8 | 3769 | 1527 | 14736 | 7294 | 15708 | 7638 |
| once | | | | | | | | |
| a day | | | | | | | | |
| 450 mg | 1.4* | 0.85* | 1771 | 803 | 12573 | 5759 | 13511 | 6134 |
| twice | | | | | | | | |
| a day | | | | | | | | |

*Tₘₐₓ values for the twice daily dosing represent the Tₘₐₓ for the first administration of the two administrations.

TABLE 6
Additional results of bioavailability study

| | Tₘₐₓ (hr) | | T_{half} (hr) | | Cₘₐₓ (ng/mL) | | | Cₗₐₛₜ (ng/mL) | | AUC (24 hr) (ng · hr/mL) | | | AUC (inf) (ng · hr/mL) | | |
|---|---|---|---|---|---|---|---|---|---|---|---|---|---|---|---|
| Dose | G. | | G. | | G. | 90% | | G. | | G. | 90% | | G. | 90% | |
| (mg) | Mean | G.C.V. | Mean | G.C.V. | Mean | CI | G.C.V. | Mean | G.C.V. | Mean | CI | G.C.V. | Mean | CI | G.C.V. |
| 900 mg | 0.9 | 74% | 15.6 | 30% | 3436 | 2877 to | 41% | 16.0 | 46% | 12668 | 10237 to | 49% | 13562 | 10956 to | 49% |
| once | | | | | | 4103 | | | | | 15678 | | | 16786 | |
| a day | | | | | | | | | | | | | | | |
| 450 mg | 1.9* | 116%* | 8.6* | 50%* | 1567 | 1312 to | 45% | 17.4 | 45% | 10972 | 8866 to | 46% | 11778 | 9516 to | 45% |
| twice | | | | | | 1872 | | | | | 13578 | | | 14579 | |
| a day | | | | | | | | | | | | | | | |

G. Mean = Geometric Mean;
G.C.V. = Geometric Coefficient of Variation;
90% CI = 90% confidence interval
*Tₘₐₓ values for the twice daily dosing represent the Tₘₐₓ for the first administration of the two administrations. T_{half} is estimated from the volume of distribution.

TABLE 7
Results of bioavailability study relating to metabolite MAT

| | Tₘₐₓ (hr) | | T_{half} (hr) | | Cₘₐₓ (ng/mL) | | | Cₗₐₛₜ (ng/mL) | | AUC (24 hr) (ng · hr/mL) | | | AUC (inf) (ng · hr/mL) | | |
|---|---|---|---|---|---|---|---|---|---|---|---|---|---|---|---|
| Dose | G. | | G. | | G. | 90% | | G. | | G. | 90% | | G. | 90% | |
| (mg) | Mean | G.C.V. | Mean | G.C.V. | Mean | CI | G.C.V. | Mean | G.C.V. | Mean | CI | G.C.V. | Mean | CI | G.C.V. |
| 900 mg | 3.9 | 26% | 13.9 | 27% | 2031 | 1850 to | 32% | 67.1 | 32% | 19328 | 17712 to | 29% | 23384 | 21524 to | 28% |
| once | | | | | | 2230 | | | | | 21091 | | | 25404 | |
| a day | | | | | | | | | | | | | | | |
| 450 mg | 13.1* | 13%* | 9.0* | 17%* | 2059 | 1875 to | 23% | 81.9 | 36% | 25319 | 23202 to | 22% | 30655 | 28217 to | 22% |
| twice | | | | | | 2260 | | | | | 27628 | | | 33304 | |
| a day | | | | | | | | | | | | | | | |

*Tₘₐₓ values for the twice daily dosing represent the Tₘₐₓ for the first administration of the two administrations. T_{half} is estimated from the volume of distribution.

TABLE 8
Results of bioavailability study relating to metabolite DAT

| | Tₘₐₓ (hr) | | T_{half} (hr) | | Cₘₐₓ (ng/mL) | | | Cₗₐₛₜ (ng/mL) | | AUC (24 hr) (ng · hr/mL) | | | AUC (inf) (ng · hr/mL) | | |
|---|---|---|---|---|---|---|---|---|---|---|---|---|---|---|---|
| Dose | G. | | G. | | G. | 90% | | G. | | G. | 90% | | G. | 90% | |
| (mg) | Mean | G.C.V. | Mean | G.C.V. | Mean | CI | G.C.V. | Mean | G.C.V. | Mean | CI | G.C.V. | Mean | CI | G.C.V. |
| 900 mg | 5.4 | 15% | 11.1 | 14% | 443 | 362 to | 64% | 22.9 | 34% | 4966 | 4160 to | 56% | 6285 | 5358 to | 51% |
| once | | | | | | 542 | | | | | 5929 | | | 7372 | |
| a day | | | | | | | | | | | | | | | |
| 450 mg | 12.4* | 21%* | 8.5* | 11%* | 451 | 369 to | 63% | 30.1 | 38% | 6311 | 5286 to | 56% | 8324 | 7097 to | 50% |
| twice | | | | | | 551 | | | | | 7534 | | | 9764 | |
| a day | | | | | | | | | | | | | | | |

*Tₘₐₓ values for the twice daily dosing represent the Tₘₐₓ for the first administration of the two administrations. T_{half} is estimated from the volume of distribution.

### Conclusion

Surprisingly, it has been found that once a day administration using the formulation of the invention provides greater bioavailability of trientine than twice a day administration using known Cuprior tablets, as can be seen by the higher AUC (24 hr), (48 hr) and (infinity) values for once a day administration. In particular, the AUC (48 hr) for once a day administration is 11600 of the AUC (48 hr) for twice a day administration. Higher bioavailability is particularly advantageous, because it could be used to decrease the amount of trientine that a particular subject needs to take to achieve the same therapeutic effect, and therefore offer a further decreased pill burden and greater improvement to treatment compliance. The observation that there are no safety concerns associated with the once a day administration, despite the higher Cₘₐₓ and total exposure (AUC) than for twice a day administration using known Cuprior tablets, is particularly beneficial. In addition, the results in Tables 7 and 8 show that the key metabolites MAT and DAT are cleared from the body after administration in a similar way after both once a day and twice a day administration.

1. A tablet dosage form comprising:
triethylenetetramine tetrahydrochloride in an amount of from 60 to 95 wt %; and
a first lubricant in an amount of from 0.1 to 10 wt %;
wherein wt % amounts are relative to the total tablet weight.

2. The tablet dosage form according to claim 1 which comprises from 70 to 95 wt % triethylenetetramine tetrahydrochloride.

3. The tablet dosage form according to claim 1, wherein the triethylenetetramine tetrahydrochloride comprises no more than 50 wt % triethylenetetramine tetrahydrochloride Form A and at least 50 wt % triethylenetetramine tetrahydrochloride Form B, wherein the wt % refers to the weight expressed as the total amount of triethylenetetramine tetrahydrochloride in the tablet dosage form.

4. The tablet dosage form according to claim 1, which comprises from 70 to 95 wt % tetrahydrochloride Form B.

5. The tablet dosage form according to claim 1, wherein the first lubricant is selected from a fatty acid or a derivative thereof, a surfactant, an inorganic material or a polymer.

6. The tablet dosage form according to claim 1, wherein:
(a) the first lubricant is selected from talc and magnesium aluminium silicate, optionally wherein the tablet dosage form comprises the first lubricant in an amount of from 0.1 to 10 wt %; or
(b) the first lubricant is selected from stearic acid, a metal stearate, sodium benzoate, a poly(ethylene oxide) and poly(propylene oxide) co-polymer, polyethylene glycol, sodium dodecyl sulfate and vegetable oil, optionally wherein the tablet dosage form comprises the first lubricant in an amount of from 0.1 to 5 wt %.

7. The tablet dosage form according to claim 1, wherein the first lubricant is a metal stearate; optionally wherein
(a) the metal stearate is one or more selected from magnesium stearate, calcium stearate and zinc stearate; and further optionally wherein the metal stearate is magnesium stearate; and/or
(b) the tablet dosage form comprises the first lubricant in an amount of from 0.1 to 0.3 wt %.

8. The tablet dosage form according to claim 1, wherein the first lubricant is magnesium stearate and wherein the tablet dosage form comprises from 0.7 to 2.0 mg magnesium stearate.

9. The tablet dosage form according to claim 1, further comprising one or more fillers in an amount of from 5 to 39.9 wt %, optionally from 5 to 29.9 wt %, relative to the total tablet weight and one or more further excipients in an amount of 0 to 10 wt % relative to the total tablet weight.

10. The tablet dosage form according to claim 9, wherein the one or more fillers comprise mannitol, lactose, cellulose, microcrystalline cellulose, calcium carbonate, sorbitol, and/or starch, optionally wherein the one or more fillers comprise mannitol.

11. The tablet dosage form according to claim 9, which comprises from 5 to 15 wt % of one or more fillers, optionally from 9 to 15 wt % of one or more fillers.

12. The tablet dosage form according to claim 1, which consists of:
(a) 70 to 95 wt % triethylenetetramine tetrahydrochloride;
0.1 to 0.3 wt % magnesium stearate;
5 to 29.9% wt % mannitol; and
0 to 10 wt % one or more further excipients,
wherein wt % amounts are relative to the total tablet weight; or
(b) 80 to 90 wt % triethylenetetramine tetrahydrochloride;
0.2 to 0.3 wt % magnesium stearate;
5 to 15 wt % mannitol; and
0 to 10 wt % one or more further excipients,
wherein wt % amounts are relative to the total tablet weight; or
(c) 80 to 85 wt % triethylenetetramine tetrahydrochloride;
0.2 to 0.3 wt % magnesium stearate;
9 to 15 wt % mannitol; and
0 to 10 wt % one or more further excipients,
wherein wt % amounts are relative to the total tablet weight.

13. The tablet dosage form according to claim 9, wherein the one or more further excipients are selected from one or more stabilizers; one or more second lubricants; and/or one or more film coating excipients; optionally wherein the one or more second lubricants is glyceryl dibehenate and/or the one or more stabilizers is colloidal silicon dioxide.

14. The tablet dosage form according to claim 13, wherein the tablet comprises the one or more second lubricants in an amount of from 0.5 to 2.5 wt % relative to the total tablet weight; and/or the tablet comprises the one or more stabilizers in an amount of from 0.1 to 0.5 wt % relative to the total tablet weight; and/or the tablet is a coated tablet and comprises one or more film coating excipients in an amount of from 0.1 to 7.5 wt % relative to the total tablet weight.

15. A tablet dosage form according to claim 1, wherein:
(a) the tablet is a breakable tablet; and/or
(b) the tablet has a hardness of from 300 to 360 N, optionally from 300 to 310 N; and/or
(c) the tablet has a friability of less than 0.15%; and/or
(d) the tablet has a disintegration time of less than 5 minutes.

16. A method of preventing or treating Wilson's disease in a subject, the method comprising administering the tablet dosage form according to claim 1 to the subject, optionally wherein the dosage form is administered to the subject once a day.

17. The tablet dosage form according to claim 10, which comprises from 5 to 15 wt % of one or more fillers, optionally from 9 to 15 wt % of one or more fillers.

18. The tablet dosage form according to claim 12, wherein the one or more further excipients are selected from one or more stabilizers; one or more second lubricants; and/or one or more film coating excipients; optionally wherein the one or more second lubricants is glyceryl dibehenate and/or the one or more stabilizers is colloidal silicon dioxide.

19. The tablet dosage form according to claim 12, wherein:
(a) the tablet is a breakable tablet; and/or
(b) the tablet has a hardness of from 300 to 360 N, optionally from 300 to 310 N; and/or
(c) the tablet has a friability of less than 0.15%; and/or
(d) the tablet has a disintegration time of less than 5 minutes.

20. A method of preventing or treating Wilson's disease in a subject, the method comprising administering the tablet dosage form according to claim 12 to the subject, optionally wherein the dosage form is administered to the subject once a day.

21. The tablet dosage form according to claim 2, which comprises from 80 to 95 wt % triethylenetetramine tetrahydrochloride.

22. The tablet dosage form according to claim 21, which comprises from 80 to 90 wt % triethylenetetramine tetrahydrochloride.

23. The tablet dosage form according to claim 22, which comprises from 80 to 85 wt % triethylenetetramine tetrahydrochloride.

24. The tablet dosage form according to claim 4, which comprises from 80 to 95 wt % triethylenetetramine tetrahydrochloride Form B.

25. The tablet dosage form according to claim 24, which comprises from 80 to 90 wt % triethylenetetramine tetrahydrochloride Form B.

26. The tablet dosage form according to claim 25, which comprises from 80 to 85 wt % triethylenetetramine tetrahydrochloride Form B.
